(19) United States
(12) Patent Application Publication
(10) Pub. No.: US 2022/0241489 A1
(43) Pub. Date: Aug. 4, 2022

(54) NEGATIVE PRESSURE DEVICE FOR ARTICULATING JOINT

(71) Applicant: Aatru Medical, LLC, Cleveland, OH (US)

(72) Inventors: John Buan, Maple Grove, MN (US); Richard L. Middaugh, Rocky River, OH (US); Timothy Wojciechowski, Westlake, OH (US); Thomas E. Lash, Chardon, OH (US)

(21) Appl. No.: 17/625,936

(22) PCT Filed: Jun. 30, 2020
(86) PCT No.: PCT/US20/40211

§ 371 Date: Jan. 10, 2022

Related U.S. Application Data
(60) Provisional application No. 62/875,210, filed on Jul. 17, 2019.

(51) Int. Cl. A61M 1/00; A61F 13/00; A61F 13/02

(52) U.S. Cl. CPC A61M 1/962; A61F 13/00068; A61F 13/0216; A61M 2205/3344; A61M 1/966; A61M 1/912; A61M 1/81

(57) ABSTRACT

A negative pressure device includes a drape covering a dressing site on a patient, a sealing element connected with the drape, a flexible gas chamber housing disposed outwardly from the drape with respect to the enclosed chamber, and a reactor. The drape is made from a thin sheet film and is capable of maintaining a negative pressure underneath the drape. The sealing element and the drape are configured so that the sealing element cooperates with the drape to define an enclosed volume covered by the drape and surrounded by the sealing element when applied to skin. The flexible gas chamber housing defines a flexible gas chamber. The reactor is positioned with respect to the enclosed volume and the flexible gas chamber to consume a gas found in air within the enclosed volume and the flexible gas chamber.

### BACKGROUND

Negative pressure therapy is a therapeutic treatment that utilizes negative pressure for skin treatments and restorative purposes. Negative pressure is a term used to describe a pressure that is below normal atmospheric pressure. Negative pressure therapy is utilized for several sites on the skin, such as a wound or an incision. Furthermore, negative pressure therapy is useful to manage wounds with complex healing concerns.

Generally, negative pressure therapy is achieved by maintaining a reduced pressure beneath a dressing on a dressing site. Typically, an electromechanical pump is connected through a hose to a dressing. The electromechanical pump draws a vacuum beneath the dressing to reduce the pressure beneath the dressing. However, these dressings can have difficulty maintaining negative pressure on an articulating joint, such as a knee or a hip. Also, the electromechanical pump makes noise and is remotely located with respect to the dressing requiring management of the hose connecting the electromechanical pump to the dressing.

### SUMMARY

In view of the foregoing, a negative pressure device includes a drape covering a dressing site on a patient, a sealing element connected with the drape, a flexible gas chamber housing disposed outwardly from the drape with respect to the enclosed chamber, and a reactor. The drape is made from a thin sheet film and is capable of maintaining a negative pressure underneath the drape. The sealing element and the drape are configured so that the sealing element cooperates with the drape to define an enclosed volume covered by the drape and surrounded by the sealing element when applied to skin. The flexible gas chamber housing defines a flexible gas chamber. The reactor is positioned with respect to the enclosed volume and the flexible gas chamber to consume a gas found in air within the enclosed volume and the flexible gas chamber.

A method for applying a negative pressure device over a dressing site includes affixing a drape and a flexible gas chamber housing defining a flexible gas chamber over the dressing site and pulling a first tab to remove a removable layer shielding a reactor from ambient to expose the reactor to air within the flexible gas chamber. The drape is capable of maintaining a negative pressure underneath the drape and cooperates with a sealing element to define an enclosed volume covered by the drape and surrounded by the sealing element. The flexible gas chamber housing is positioned over the drape so that the flexible gas chamber is in fluid communication with the enclosed volume. The reactor is configured to consume a gas found in air within the enclosed volume and the flexible gas chamber when exposed to air in the flexible gas chamber.

### BRIEF DESCRIPTION OF THE DRAWINGS

FIG. 1 is an exploded perspective view of a negative pressured device according to an embodiment.

FIG. 2 is a perspective cross-sectional view of the negative pressure device depicted in FIG. 1.

FIG. 3 is a perspective view of the negative pressure device having a differently shaped drape than that shown in FIGS. 1 and 2.

FIG. 4 is a top view of an alternative sealing element for use with the negative pressure device depicted in FIG. 1.

### DETAILED DESCRIPTION

The invention is not limited in its application to the details of construction and arrangement of components provided in the following description or illustrated in the attached drawings. The invention is capable of other embodiments and being practiced in various manners. The phraseology and terminology used herein is for the purpose of description and should not be regarded as limiting. Moreover, the use of “including,” “comprising,” or “having” and variations thereof is meant to encompass the items listed thereafter and equivalents thereof as well as additional items.

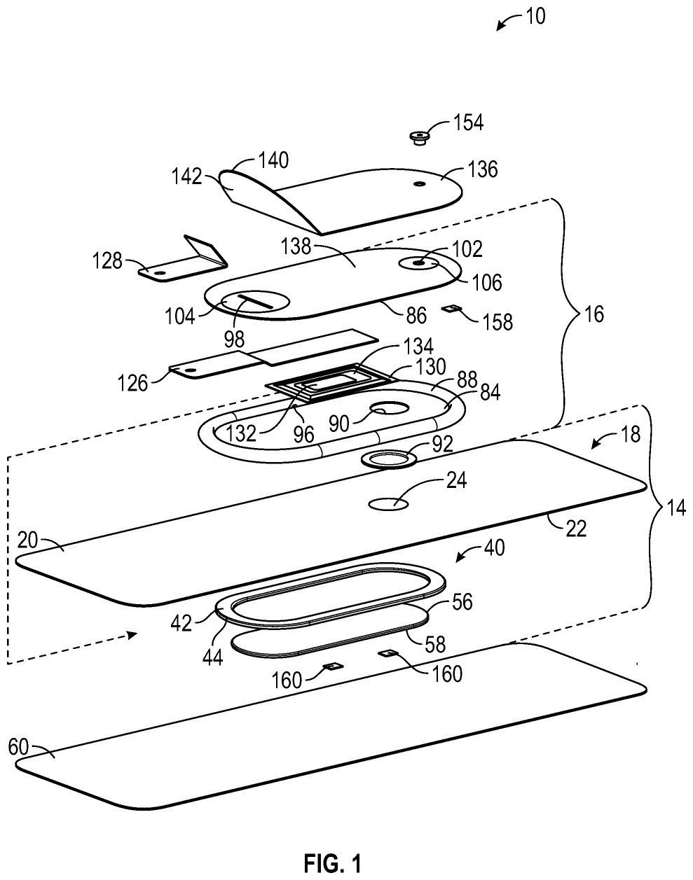

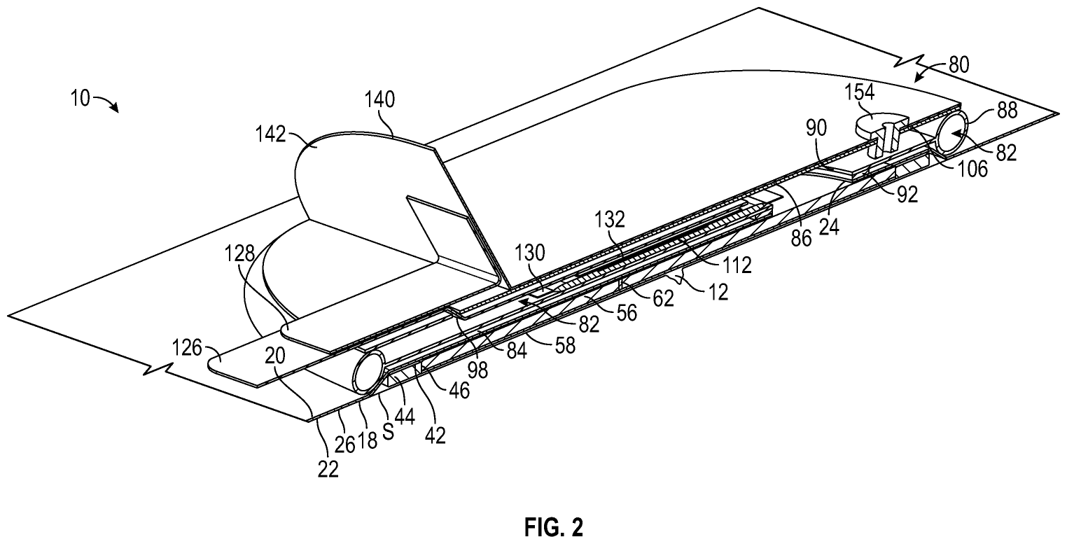

FIGS. 1 and 2 depict an embodiment of the negative pressure device 10 that is useful to create a pressure that is below normal atmospheric pressure at a dressing site 12 (FIG. 2). Negative pressure described herein is pressure below atmospheric pressure. The dressing site 12 can be a wound, an incision, or any other portion of a patient in which negative pressure therapy is to be applied. In the embodiment depicted in FIGS. 1 and 2, the negative pressure device 10 includes at least one dressing 14 and a vacuum source 16.

With reference to FIGS. 1 and 2, the dressing 14 includes a drape 18 that covers the dressing site 12. The dressing 14 is capable of maintaining a negative pressure environment on the dressing site 12 beneath the drape 18 and around the dressing site 12 for extended periods of time, and also allows easier handling for placement of the dressing 14 onto the skin S (FIG. 2). The drape 18 is a thin sheet film capable of maintaining a negative pressure underneath the drape 18 when sealed against the skin S upon application of negative pressure from the vacuum source 16. The thin film from which the drape 18 is made can be substantially impermeable to liquids but somewhat permeable to water vapor, while still being capable of maintaining negative pressure underneath the drape 18. For example, the thin film material from which the drape 18 is made may be constructed of polyurethane or other semi-permeable material such as that sold under the Tegaderm® brand or 9834 TPU tape available from 3M. Similar films are also available from other manufacturers. Although the film from which the drape 18 is made may have a water vapor transmission rate of about 836 g/m²/day or more, these films are still capable of maintaining a negative pressure underneath the drape 18 when an appropriate seal is made around the periphery of the dressing site 12. The drape 18 can also be configured to allow a selected gas diffuse through the drape. Alternatively, the drape 18 can be occlusive. Furthermore, the drape 18 can be transparent or translucent to allow a user to view underneath the drape 18.

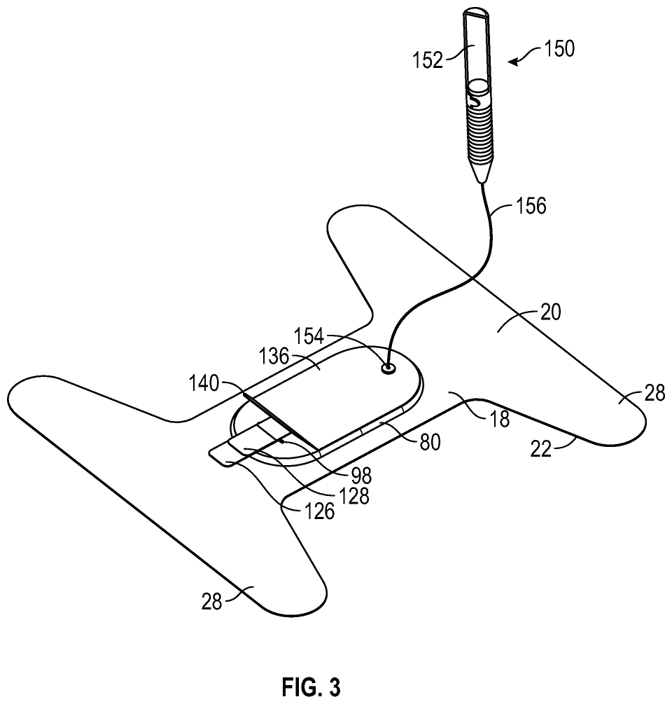

The drape 18 includes a drape top surface 20 and a drape bottom surface 22. The drape 18 further includes at least one opening 24. An adhesive layer 26 is disposed across the bottom surface 22 of the drape 18 to facilitate attachment of the drape 18 to the skin S and to facilitate assembly of the dressing 14. The adhesive of the adhesive layer 26 can be a pressure-sensitive acrylic-based adhesive. With reference to FIG. 3, in another embodiment, the drape 18 may take other configurations and may include elongated straps 28. The straps 28 may help secure the dressing 14 to the dressing site 12 during articulating motions. As such, the dressing 14 is secured to maintain its position over the dressing 14 over an articulating joint during articulating movements. The drape 18 can be made in a variety of shapes and sizes to cover a variety of dressing sites 12.

The dressing 14 may further include a first sealing element 40 placed underneath the drape 18 and surrounding the opening 24. When the dressing 14 is affixed to the skin S, the first sealing element 40 is positioned to surround the dressing site 12. The first sealing element 40 cooperates with the drape 18 to define an enclosed volume 46 covered by the drape 18 and surrounded by the first sealing element 40 when the dressing 14 is applied to skin S. The first sealing element 40 functions like a gasket in that the first sealing element 40 prevents fluid (including air) from entering or escaping the enclosed volume 46 between the drape 18 and skin S. The first sealing element 40 is distinct from the adhesive layer 26. When properly sealed, air or select gases found in air can selectively exit the enclosed volume 46 of the dressing 14 through the opening 24. The first sealing element 40 helps maintain negative pressure within the dressing 14. The first sealing element 40 can be made from a material such as a silicone, hydrocolloid or a hydrogel material.

In the illustrated embodiment, the first sealing element 40 includes a sealing backing film 42 having a silicone gel 44 deposited thereon. The sealing backing film 42 is useful to affix the silicone gel 44 to the bottom surface 22 of the drape 18. In an embodiment where the first sealing element 40 includes the silicone gel 44, the sealing backing film 42 can be a polyurethane, polyethylene, polypropylene, or co-polyester film, that is brought in contact with the adhesive layer 26 on the bottom surface 22 of the drape 18 to fix the first sealing element 40 to the drape 18. Typically, silicone does not bond well to an acrylic-based adhesive and the pressure-sensitive acrylic-based adhesive makes up the adhesive layer 26 of the drape 18 in the illustrated embodiment. By providing the silicone gel 44 on the sealing backing film 42, the silicone gel 44 can be fixed with respect to the drape 18 while still being able to utilize a pressure-sensitive acrylic-based adhesive as the adhesive layer 26 on the drape 18. This allows for the benefit of providing the silicone gel 44 with the dressing 14 that can contact the skin S around the dressing site 12 and provide a much better seal than only having the pressure-sensitive acrylic-based adhesive, which allows for negative pressure to be obtained underneath the drape 18 around the dressing site 12.

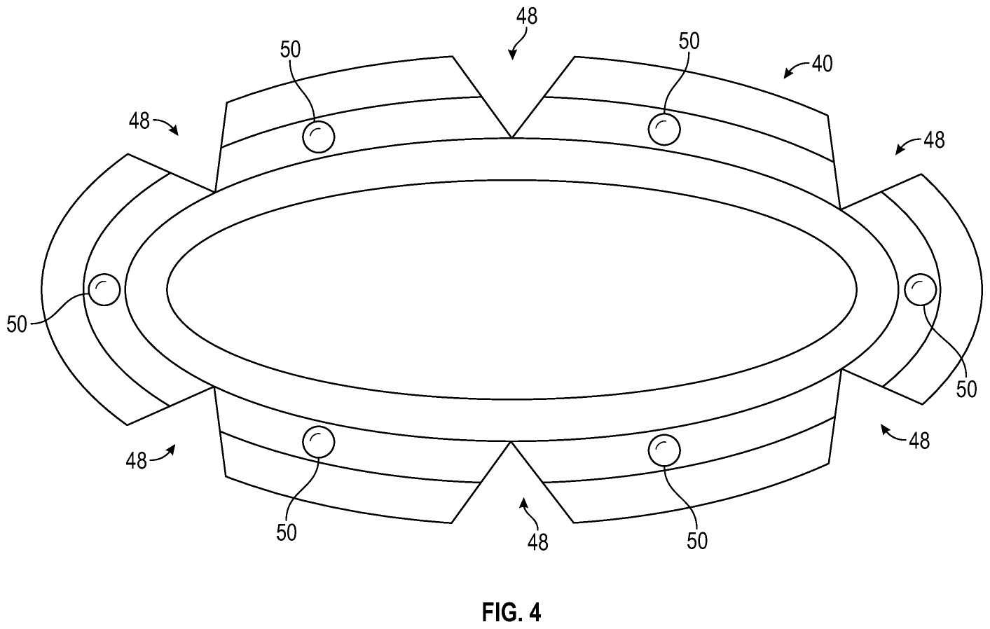

In an embodiment depicted in FIG. 4, the first sealing element 40 can include a plurality of cuts 48 in the shape of a “V” (referred to hereinafter as “V-cuts”) on an outer periphery of the first sealing element 40. The V-cuts 48 are made on an outer perimeter of the sealing element 40 to maintain a vacuum seal in the full therapeutic ranges for articulating motions and remain flexible and conforming from 90-180 degrees of motion and 180-360 degrees of motion. Alternatively, the V-cuts could also be made on an inner perimeter, and both the outer and inner perimeters could have notches, that would make a Z profile around the perimeter, which would be very flexible to tension and compression. Also, the notches could take shapes other than V-cuts. With reference back to the embodiment depicted in FIG. 4, the V-cuts 48 allow the first sealing element 40 to maintain a vacuum tight seal when bent in full circle. In other words, the V-cuts 48 allow the first sealing element 40 to maintain a vacuum tight seal when the first sealing element 40 is bent over. Thus, the V-cuts 48 permit the first sealing element 40 and the dressing 14 to maintain negative pressure for the duration of articulating motions ranging from 180-90 degrees of motion or less. Therefore, when the dressing 14 is placed over the joint, the first sealing element 40 maintains its seal as the patient bends and straightens the joint. In another embodiment, the V-cuts 48 are also made in the sealing backing film 42. The first sealing element 40 may further include small dimples and/or pinholes 50. The small dimples 50 add suction and hold the first sealing element 40 tight to the skin S when the small dimples 50 are pressed into place. In one embodiment, the first sealing element 40 is formed and shaped as concentric rings which maintain a tight seal to the dressing site 12, as depicted in FIG. 4. The concentric rings are flexible to maintain their seal throughout articulating motions.

With reference back to FIGS. 1 and 2, the dressing 14 may also include a wicking or absorbing element 56. The wicking element 56 is applied onto the bottom surface 22 of the drape 18 and is surrounded by the sealing element 40. In the illustrated embodiment, the wicking element 56 is affixed to the drape 18 via the pressure-sensitive acrylic-based adhesive of the adhesive layer 26. The wicking element 56 is made from an absorbent material that is capable of absorbing exudate from the dressing site 12. The wicking element 56 can be made from super absorbent polymers, absorbent beads, foams, or natural absorbents. Also, the wicking element 56 can provide appropriate voids for gases found in air so that reduced pressure can be maintained. For example, the wicking element 56 can be made from a relatively rigid foam so that gas voids are maintained while absorbing exudate from the dressing site 12. The wicking element 56 can also be made from superabsorbent polymers that expand and form at least one gas void, for example, between adjacent beads, to provide aforementioned volume control. The wicking element 56 can also be a hydroactive wound pad available under the trademark Vilmed®, which chemically absorbs exudate and precludes exudate from passing through the wicking element 56 toward the vacuum source 16 unlike a sponge. A silicone coating 58 can be provided on the skin-contacting side of the wicking element 56, if desired, which is very compatible with skin and other tissue. The dressing 14 may further comprise another type of wound contact layer other than the silicone coating 58. Such a wound contact layer can be made from an elastomeric material, such as a polymeric material that has rubber-like properties. Furthermore, the wound contact layer can be an elastomeric material that is a thin, flexible elastomeric film. Some examples of such material include a silver coated nylon, a perforated silicone mesh, or other materials that will not stick to the patient's tissue. The silicone coating 58 or other wound contact layer contacts the dressing site 12. Additionally, the wound contact layer can include at least one opening to cooperate with the wicking element 56 so that the wicking element 56 retains exudate traveling from the dressing 14 into the enclosed volume 46.

Furthermore, the wicking element 56 may include a slit 62 (only depicted in FIG. 2) that extends from a bottom surface of the wicking element 56 to a top surface of the wicking element 56.

With reference to FIG. 1, a release liner 60 is be disposed on the bottom surface 22 of the drape 18, and is removed before the dressing 14 is applied to the dressing site 12. The release liner 60 can have a larger area than the dressing 14. The release liner 60 can be made as one piece or multiple pieces. The release liner 60 also covers underneath the silicone coating 58, the alternative wound contact layer, and the first sealing element 40. When the release liner 60 is removed, the adhesive layer 26 on the drape 18 and the first sealing element 40 are exposed. After the release liner 60 is removed, the dressing 14 is placed on the patient, and the adhesive layer 26 secures the dressing 14 to the patient's skin S around the dressing site 12. Oftentimes, release liners are coated with a silicone coating; however, silicone coatings often are not compatible with silicone gel which can result in the silicone gel being pulled along with the release liner 60 when the release liner 60 is removed from the drape 18 and other components of the dressing 14.

In the illustrated example, the release liner 60 is coated with a fluoropolymer release coating on the side of the release liner 60 that contacts the pressure-sensitive acrylic-based adhesive of the adhesive layer 26 on the drape 18 and the appropriate surfaces of the silicone gel 44 of the first sealing element 40 and the wicking element 56. The release liner 60 can be a polyester film coated on one side with the fluoropolymer release coating, which can be used with silicone adhesives. This release coating is also compatible with the pressure-sensitive acrylic-based adhesive on the bottom surface 22 of the drape 18 such as that available with 9834 TPU tape available from 3M.

With further reference to FIG. 2, a flexible gas chamber housing 80 is disposed outwardly from the drape 18 with respect to the enclosed volume 46. The flexible gas chamber housing 80 defines a flexible gas chamber 82 that is in fluid communication with the enclosed volume 46. The flexible gas chamber housing 80 includes a lower layer 84 and an upper layer 86, which can be made from thin materials similar to the drape 18 that when sealed are capable of maintaining a negative pressure with respect to atmosphere. Either the lower layer 84 or the upper layer 86 can also be made from thicker materials such as rubber-like materials, vinyl and the like that when sealed are capable of maintaining a negative pressure with respect to atmosphere.

A flexible hose 88 is attached to the lower layer 84 and the upper layer 86 to enclose and define a portion of the flexible gas chamber 82. The flexible hose 88 can be made in a various diameters to increase or decrease the volume of the flexible gas chamber 82 and manage partial negative pressure under the dressing 14. The lower layer 84, the upper layer 86 and the flexible hose 88 are configured to twist and bend to conform to the curvatures of a body during articulating motions without greatly changing the internal area of the flexible gas chamber 82.

The lower layer 84 further includes a lower opening 90 that is the same size as the opening 24 in the drape 18 in the illustrated embodiment. When the flexible gas chamber housing 80 is attached with the dressing 14, the lower opening 90 in the lower layer 84 is aligned with the opening 24 in the drape 18. A second sealing element 92, which can be similar to the first sealing element 40, is disposed around the opening 24 in the drape 18, the lower opening 90 in the lower layer 84 and between the drape 18 and the lower layer 84. A bottom surface of the second sealing element 92 is affixed to the top surface 20 of the drape 18, and a top surface of the second sealing element 92 is affixed to the lower layer 84, as depicted in FIG. 2. The second sealing element 92 may also include adhesive on the top surface for securing to the lower layer 84 and/or adhesive on the bottom surface for securing to the drape 18. The second sealing element 92 functions similarly to the first sealing element 40 and prevents fluid (including air) from entering or escaping between the drape 18 and the lower layer 84.

When properly sealed, air or select gases found in air can selectively exit the enclosed volume 46 and enter the flexible gas chamber 82 through the opening 24 and the lower opening 90. The flexible hose 88 also includes a hose opening 96 (see FIG. 1), or multiple hose openings so that the internal volume of the flexible hose 88 can also operate as part of the flexible gas chamber 82.

With reference back to FIG. 1, the upper layer 86 includes an opening, which is in the form of an elongated slit 98 in the illustrated embodiment. When not covered, the elongated slit 98 exposes the flexible gas chamber 82 and the enclosed volume 46 to ambient. The upper layer 86 may further include a valve opening 102. In the illustrated embodiment, the valve opening 102 is disposed on the opposite end of the upper layer 86 as the elongated slit 98. The upper layer 86 can be a flexible film, and in such an embodiment, the upper layer 86 can include a first relatively more rigid section 104 disposed around the slit 98, and a second relatively more rigid section 106 around the valve opening 102.

In the illustrated embodiment, the vacuum source 16 includes a reactor 112 configured to react with a selected gas found in air, e.g., oxygen, to provide reduced pressure to the enclosed volume 46 when in fluid communication with the enclosed volume 46. An example of a reactor 112 is described in US 2014/010989A1. US 2014/0109890A1 describes an oxygen based heater; however, the oxygen based heater can be used as the reactor to consume oxygen within the enclosed volume 46 thus producing a partial vacuum within the enclosed volume 46. The reactor 112 is positioned in the flexible gas chamber 82 in the illustrated embodiment. In an alternative embodiment, the reactor 112 could be positioned beneath the drape 18.

The vacuum source 16 could also be a zinc/air cell. When the vacuum source 16 is a zinc/air cell, the zinc/air cell can react with oxygen found in the enclosed volume 46 and the flexible gas chamber 82 thus reducing the pressure in the enclosed volume 46. In the embodiment where the vacuum source 16 is a zinc/air cell, the zinc/air cell is disposed in the flexible gas chamber 82, and the upper layer 86 covers the zinc/air cell. A circuit (not shown) having a normally open switch can be connected to an anode and cathode, respectively, on the zinc/air cell. An operator can depress the upper layer 86, for example, in the vicinity of the switch to close the circuit. The zinc/air cell reacts with oxygen in the enclosed volume 46 and the flexible gas chamber 82 to remove the oxygen from the enclosed volume 46 and thus reduce the pressure within the enclosed volume 46.

In lieu of the reactor and zinc/air cell described above, the vacuum source 16 may include one or any combination of a plunger and piston or syringe capable of drawing a vacuum, electro-chemical pumps, vacuum-on-demand devices (referred to herein as VOD), electrolyzers, pressure-reducing solid state devices, oxygen absorbing iron packets, or getters of zirconium titanium, vanadium, iron, lithium, lithium metal, magnesium, calcium, lithium barium combinations, zinc/air battery, zinc/air battery components, or other materials highly reactive with the selected gases, for example, nitrogen, carbon dioxide, and oxygen gasses found in wound bed environments.

In the embodiment in which the vacuum source is the reactor 112, at least one pull tab extends from the flexible gas chamber 82 to ambient through the slit 98 in the upper layer 86. In one embodiment, the at least one pull tab includes a first pull tab 126 and a second pull tab 128. In one embodiment, the first pull tab 126 and the second pull tab 128 are separate elements, whereas, in another embodiment, the first pull tab 126 and the second pull tab 128 could be connected or integral.

A packet 130 including a removable layer 132 covers the reactor 112 so as to prevent the reactor 112 from being exposed to ambient until after the removal of the removable layer 132 from the packet 130. The packet 130 can be a foil packet that is hermetically sealed around the reactor 112. The first pull tab 126 extends through the slit 98 and is connected to the removable layer 132. The first pull tab 126 can be pulled to remove the first pull tab 126 from the slit 98. When the first pull tab 126 is pulled through the slit 98, the removable layer 132 is removed from the packet 130 and, if desired, from the flexible gas chamber 82 through the slit 98, exposing the reactor 112 to air within the enclosed volume 46 and the flexible gas chamber 82. After the removal of the removable layer 132, the reactor beings to react with a selected gas, e.g. oxygen, in the flexible gas chamber 82 and the enclosed volume 46.

In the illustrated embodiment, the packet 130 is affixed to the lower layer 84 by an adhesive (not shown). The removable layer 132 is coated on a bottom surface with adhesive, with the exception of a small section 134 at an end of the removable layer 132 opposite from the slit 98. The first pull tab 126 connects with the small section 134, which lacks the adhesive, and the connection between the first pull tab 126 and the removable layer 132 is limited to the small section 134 in that the first pull tab 126 is free to move with respect to the remainder of the removable layer 132 that carries the adhesive on the bottom surface of the removable layer 132 and is affixed to the packet 130. As such, when the first pull tab 126 is pulled away from the upper layer 86 through the slit 98, the removable layer 132 rolls over on itself as the removable layer 132 is peeled away from the packet 130.

The second pull tab 128 is connected to a thin film 136, which is placed over and adhered to a portion of a top surface 138 of the upper layer 86. The thin film 136 includes a flap 140 and, as depicted in FIG. 2, the slit 98 is disposed underneath the flap 140. The second pull tab 128 is connected to a release layer 142 provided on a bottom surface of the thin film 136. The release layer 142 covers an adhesive on a bottom surface of the flap 140. When the second pull tab 128 is pulled, the second pull tab 128 removes the release layer 142 from the flap 140, and the adhesive disposed on the bottom surface of the flap 140 is exposed. The flap 140 can then moved toward the upper layer 86 to cover the top surface 138 of the upper layer 86 (and the first relatively more rigid section 104 disposed around the slit 98, if provided) and thus also covers the slit 98. The flap 140 is secured to the upper layer 86 by the adhesive disposed on the bottom surface of the flap 140. In result, the flexible gas chamber 82 and the enclosed volume 46 are no longer exposed to ambient via the slit 98. When the thin film 136 covers the slit 98, the reactor 112 reacts with the selected gas found in the flexible gas chamber 82 and the enclosed volume 46. Reduced pressure is therefore developed in the flexible gas chamber 82 and the enclosed volume 46. Either the first pull tab 126 or the second pull tab 128 can be pulled first and the tabs are referred to as “first” and “second” to differentiate them from one another and not to connote a particular order of operation.

When the flexible gas chamber 82 and the enclosed volume 46 are under negative pressure, the thin film 136 is drawn in through the slit 98 toward the flexible gas chamber 82. As such, the thin film 136 cooperating with the slit 98 can provide an indication to the user that the flexible gas chamber 82 and the enclosed volume 46 are under negative pressure. An indicator, e.g., lines, a cross, or the like, can also be provided on the thin film 136 in the vicinity of the slit 98 to provide further indication or negative pressure.

With reference to FIG. 3, a mechanical pump assembly 150 having a pump chamber 152 can connect to the flexible gas chamber housing 80 via a valve 154. An example of the mechanical pump assembly 150 is described in U.S. application Ser. No. 15/798,777 and PCT/US19/12298. The mechanical pump assembly 150 can also connect with the valve 154 via a hose 156, or the mechanical pump assembly 150 can connect directly to the first valve 154 eliminating the hose 156. The valve 154 is disposed in the valve opening 102 in the upper layer 86. The valve 154 could be located elsewhere, e.g., in or along the hose 156. When the mechanical pump assembly 150 is connected to the valve 154, the pump chamber 152 in the mechanical pump assembly 150 is in fluid communication with the flexible gas chamber 82 and the enclosed volume 46 via the valve 154. Actuation of the mechanical pump assembly 150 draws air from the enclosed volume 46 and the flexible gas chamber 82 through the valve opening 102, the valve 154, and the hose 156 (if provided) into the pump chamber 152 of the mechanical pump assembly 150.

The valve 154 can have two operating states. In the first, e.g., open, operating state, the valve 154 allows air to exit the enclosed volume 46 and the flexible gas chamber 82 through the valve 154 and into the pump chamber 152 of the mechanical pump assembly 150 when the mechanical pump assembly 150 and/or the hose 156 is inserted into or operatively connected with the valve 154. In the second, e.g. closed, operating state, the valve 154 precludes ambient air from entering the flexible gas chamber 82 and the enclosed volume 46 through the valve opening 102 and the valve 15. Examples of such valves include, but are not limited to, a spring-biased check valve, a valve having flaps, e.g., a duckbill valve, and valve having a silicone dome with right-angled slits cut in the top of the dome.

The negative pressure device 10 can be susceptible to reaching a negative pressure below the target pressure range, e.g. too much vacuum or negative pressure may be achieved in the flexible gas chamber 82 and the enclosed volume 46. In order to maintain the target pressure range, the valve 154 can operate as a relief valve to release pressure as needed. In an alternative arrangement, the relief valve can be in addition to the aforementioned valve 154. The relief valve can be any valve that can manually or automatically release pressure as needed. In another embodiment, the relief valve is disposed on the drape 18 of the dressing 14. It is to be understood that the relief valve functions similarly in an embodiment in which the relief valve is disposed on the dressing 14 as the relief valve disposed on the upper layer 86. As a pressure differential between ambient and the enclosed volume 46 of the dressing 14 moves outside of a predetermined pressure range, which can be set for example between −50 mmHg and −200 mmHg with respect to atmosphere, the valve 154 opens and air from ambient enters the flexible gas chamber 82 and the enclosed volume 46 until the internal pressure reaches the pressure at which the valve 154 reseals and closes. The flexible gas chamber 82 and the enclosed volume 46 are then subject to the amount of negative pressure at which the valve reseals, which can be different than the pressure differential at which the valve 154 is opened while still being within the therapeutic range, e.g. between −50 mmHg and −200 mmHg with respect to atmosphere.

In another embodiment, the valve 154 disposed in the upper layer 86 can be a bidirectional valve. The bidirectional valve can be similar construction to the valve described in U.S. Pat. No. 5,439,143. The mechanical pump assembly 150 can be in fluid communication with the flexible gas chamber 82 and the enclosed volume 46 through the bidirectional valve. The bidirectional valve may include three operating states. In the first operating state, gas is allowed to exit the flexible gas chamber 82 and the enclosed volume 46 through the bidirectional valve when the external pressure is below the flexible gas chamber 82 and the enclosed volume 46. In the second operating state, the bidirectional valve precludes gas from entering or exiting the flexible gas chamber 82 and the enclosed volume 46 through the bidirectional valve when the pressure of the flexible gas chamber 82 and the enclosed volume 46 is between a first predetermined threshold and a second predetermined threshold. In the third operating state, the bidirectional valve allows gas from ambient to enter the flexible gas chamber 82 and the enclosed volume 46 through the bidirectional valve when the pressure in the flexible gas chamber 82 and the enclosed volume 46 is below the predetermined threshold. In one embodiment, the predetermined threshold is 560 mmHg or 200 mmHg below atmospheric pressure.

With reference back to FIG. 1, the dressing 14 may further include at least one pressure sensor 158 for sensing the pressure within the dressing 14. The pressure sensor 158 can include RFID capabilities to communicate with wireless technologies, such as, but not limited to, a smartphone. The pressure sensor 158 can also use wireless and touchless WiFi technology to measure the negative pressure underneath the dressing 14 and communicate with a patient or caregiver. These communications can be transferred to a computer, tablet, smartphone, or digital assistant. In one embodiment, the pressure sensor 158 communicates via Amazon's Alexa®. An alarm can also be used to alert the patient or caregiver that the negative pressure in the enclosed volume 46 is outside a predetermined threshold. The dressing 14 may further include one or more small electrodes 160 positioned proximate the dressing site 12. In one embodiment, the small electrodes 160 are positioned along each side of an incision. Alternatively, the small electrodes 160 are positioned parallel to the incision. The small electrodes 160 are configured to deliver electro-stimulation to the dressing site 12 on demand. Power to the pressure sensor 158 and the small electrodes 160 can be provided from a variety of sources, such as zinc air batteries, a chemical pump and zinc air batteries, or a zinc air battery printed on the drape 18.

A method for applying the negative pressure device 10 over the dressing site 12 will now be described. Although the figures may show a specific order of method steps, the order of the steps may differ from what is depicted. Also two or more steps may be performed concurrently or with partial concurrence. All such variations are within the scope of the disclosure.

The drape 18 and the flexible gas chamber housing 80 defining a flexible gas chamber 82 are placed over the dressing site 12 to cover the dressing site 12. The drape 18 and the flexible gas chamber housing 80 can be already connected with one another prior to placement over the dressing site 12.

The first pull tab 126 extending through the slit 98 disposed on the upper layer 86 of the flexible gas chamber 82 is pulled to remove the removable layer 132 shielding the reactor 112 from ambient. In result, the reactor 112 is exposed to air and begins to react with the selected gas, e.g. oxygen, in the flexible gas chamber 82 and the enclosed volume 46. Then, the second pull tab 128 can be pulled to expose the adhesive on the bottom surface of the flap 140 on the thin film 136 disposed on the top surface of the upper layer 86. The flap 140 is pressed onto the top surface 138 of the upper layer 86 to cover the slit 98 and secured by the adhesive disposed on the bottom surface of the flap 140. In result, the flexible gas chamber 82 and the enclosed volume 46 are no longer exposed to ambient. When the flexible gas chamber 82 and the enclosed volume 46 are under negative pressure, the thin film 136 provides an indication to the user by drawing in through the slit 98 toward the flexible gas chamber 82. If the negative pressure device 10 reaches a negative pressure below the target pressure range, the valve 154 can open to allow air to enter the enclosed volume 46 and the flexible gas chamber 82.

It will be appreciated that various of the above-disclosed embodiments and other features and functions, or alternatives or varieties thereof, may be desirably combined into many other different systems or applications. Additionally, even though the invention has been described with reference to specific exemplifying embodiments thereof, many different alterations, modifications and the like will become apparent for those skilled in the art. Variations to the disclosed embodiments can be understood and effected by the skilled artisan in practicing the claimed invention, from a study of the drawings, the disclosure, and the appended claims. Furthermore, in the claims, the word “comprising” does not exclude other elements or steps, and the indefinite article “a” or “an” does not exclude a plurality. Also that various presently unforeseen or unanticipated alternatives, modifications, variations or improvements therein may be subsequently made by those skilled in the art which are also intended to be encompassed by the following claims.

1. A negative pressure device comprising:
a drape for covering a dressing site on a patient, the drape being made from a thin sheet film and capable of maintaining a negative pressure underneath the drape;
a sealing element connected with the drape, the sealing element and the drape being configured such that when applied to skin the sealing element cooperates with the drape to define an enclosed volume covered by the drape and surrounded by the sealing element;
a flexible gas chamber housing disposed outwardly from the drape with respect to the enclosed volume and defining a flexible gas chamber; and
a reactor positioned with respect to the enclosed volume and the flexible gas chamber so as to consume a gas found in air within the enclosed volume and the flexible gas chamber.

2. The negative pressure device of claim 1, wherein the sealing element is formed as concentric rings.

3. The negative pressure device of claim 1 or 2, wherein the sealing element comprises a sealing backing film affixed to a bottom surface of the drape, and silicone positioned on the sealing backing film.

4-17. (canceled)

18. A method for applying a negative pressure device over a dressing site comprising:
affixing a drape and a flexible gas chamber housing defining a flexible gas chamber over the dressing site, wherein the drape is capable of maintaining negative pressure underneath the drape and cooperates with a sealing element to define an enclosed volume covered by the drape and surrounded by the sealing element and the flexible gas chamber housing is positioned over the drape with the flexible gas chamber in fluid communication with the enclosed volume; and
pulling a first tab to remove a removable layer shielding a reactor from ambient to expose the reactor to air within the flexible gas chamber, the reactor configured to consume a gas found in air within the enclosed volume and the flexible gas chamber when exposed to ambient.

19. The method of claim 18, further comprising:
connecting a mechanical pump assembly having a pump chamber to the flexible gas chamber housing so that the pump chamber is in fluid communication with the enclosed volume and the flexible gas chamber.

20. The method of claim 18 or 19, further comprising:
pulling a second tab to expose adhesive on a film and covering a slit in the flexible gas chamber housing through which the first tab was pulled after removing the first tab from the slit.

21. The negative pressure device of claim 1, wherein the sealing element comprises dimples configured to add suction and hold the sealing element tight to the skin when pressed into place.

22. The negative pressure device of claim 1, wherein the flexible gas chamber housing includes a lower layer and a lower opening disposed in the lower layer.

23. The negative pressure device of claim 1, wherein the flexible gas chamber housing includes an upper layer including a slit, and the negative pressure device further comprises:
a first pull tab which extends from the flexible gas chamber to ambient through the slit, and
a packet including a removable layer connected to the first pull tab which shields the reactor from ambient until the removable layer is removed from the packet.

24. The negative pressure device of claim 23, wherein the upper layer includes a flexible film and at least one relatively rigid section disposed around the slit.

25. The negative pressure device of claim 24, wherein the upper layer includes a flexible hose connected with the upper layer and having a hose opening, wherein an internal volume of the flexible hose operates as part of the flexible gas chamber.

26. The negative pressure device of claim 1, further comprising a wicking element for absorbing fluid from the dressing site.

27. The negative pressure device of claim 1, further comprising at least one pressure sensor having RFID capabilities to communicate the pressure of the enclosed volume wirelessly.

28. The negative pressure device of claim 1, further comprising at least one electrode for delivering electro-stimulation to the dressing site.

29. The negative pressure device of claim 1, further comprising a valve having two operating states wherein,
the valve is configured to allow gas to exit from the enclosed volume through the valve in a first operating state, and
the valve is configured to remain closed so as to prevent air from entering or exiting the enclosed volume through the valve in a second operating state.

30. The negative pressure device of claim 29, wherein the valve includes a third operating state in which gas from ambient is able to enter the enclosed volume through the valve when a pressure differential between ambient and the enclosed volume is outside a predetermined pressure range.

31. The negative pressure device of claim 30, further comprising a mechanical pump assembly including a pump chamber fluidly connectable to the enclosed volume via the valve, and configured to draw air from the enclosed volume into the pump chamber when fluidly connected with the enclosed volume.

32. The negative pressure device of claim 29, wherein the flexible gas chamber housing includes an upper layer including a valve opening, which receives the valve, and at least one relatively rigid section disposed around the valve opening.

33. The negative pressure device of claim 1, further comprising a relief valve located on the flexible gas chamber housing or the drape and in selective fluid communication with the enclosed volume, the flexible gas chamber and ambient, the relief valve being configured to allow gas from ambient to enter the enclosed volume through the relief valve when a pressure differential between ambient and the enclosed volume is outside a predetermined pressure range.

34. The negative pressure device of claim 1, wherein the reactor is disposed in the flexible gas chamber.

35. The negative pressure device of claim 1, wherein the sealing element is a ring-shaped gasket.

36. The negative pressure device of claim 1, wherein the sealing element is plurality of concentric ring-shaped gaskets.
